(19) United States
(12) Patent Application Publication
(10) Pub. No.: US 2002/0162551 A1
(43) Pub. Date: Nov. 7, 2002

(54) Cymbal-shaped actuator for a nebulizing element

(76) Inventor: Craig M. Litherland, Cupertino, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/848,088

(22) Filed: May 2, 2001

(51) Int. Cl. A61M 11/00

(52) U.S. Cl. 128/200.14

(57) ABSTRACT

An aerosol generator comprises a vibratable element and a support member that is coupled to the vibratable element. The support member is cymbal-shaped and has a concave surface and a central opening, and the concave surface faces the vibratable element. A vibratable member is disposed across the central opening and has a plurality of apertures. In this way, liquid that is applied to a rear surface of the vibratable member is ejected from the front surface as an atomized spray upon vibration of the vibratable element.

### BACKGROUND OF THE INVENTION

This invention relates generally to the field of aerosolization, and in particular to the production of fine liquid droplets. More specifically, the invention relates to aerosol generators and methods for producing fine liquid droplets with high flow rates or efficiencies.

The ability to aerosolize or nebulize small liquid droplets is important to a variety of industries. Merely by way of example, many pharmaceuticals can now be delivered to the lungs in liquid form. Aerosolization is also a useful technique to dispense deodorizers, perfumes, insecticides or the like into the atmosphere.

In some cases it may be desirable to achieve a high flow rate through the aerosol generator so that larger volumes of liquid may be aerosolized in a shorter time. For example, when aerosolizing a unit dosage of a medicament, it may be desirable to rapidly aerosolize the entire dose so that the user may inhale the entire dose with on inhalation.

Hence, this invention relates to the operation of aerosol generators at high efficiencies to increase the flow rate of the aerosolized liquid.

### SUMMARY OF THE INVENTION

In one embodiment, an aerosol generator comprises a vibratable element having a central opening. A support member is coupled to the vibratable element and also includes a central opening. A vibratable member is disposed across the opening of the support member and has a front surface, a rear surface, and a plurality of apertures extending between the two surfaces. In certain vibrational modes, the support member is configured to move the vibratable member in substantially rigid body motion such that the vibratable member does not appreciably flex.

In operation, liquid is supplied to the rear surface of the vibratable member, and the vibratable element is vibrated. In certain vibrational modes, the vibratable member is generally translated back and forth without appreciable flexing. This causes liquid droplets to be ejected from the front surface of the vibratable member as the vibratable member is accelerated. By vibrating the vibratable member in this manner, essentially all of the apertures may produce liquid droplets, resulting in a high flow rate of liquid through the aerosol generator. In this way, the efficiency of the aerosol generator is greatly increased. In other vibrational modes, some flexing of the vibratable member may occur, but will be within acceptable limits so that sufficient volumes of liquid are aerosolized.

In one aspect, such rigid body motion is achieved by configuring the support member to have a generally concave surface that generally faces the vibratable element. In another aspect, the vibratable member may be annular in geometry, such as an annular piezoelectric transducer or crystal. The annular vibratable member is coupled to an outer periphery of the support member so as to be concentric with the vibratable member. Further, the vibratable element is configured to radially expand and contract to translate the vibratable member in a direction that is generally perpendicular to the radial movement of the vibratable member.

In one particular aspect, the support member may be cymbal or hat shaped and have a flat outer region, an angled intermediate region and a flat inner region that contains the central opening. As the vibratable element (which is attached to the outer region) is vibrated, the inner region is generally translated back and forth to accelerate the vibratable member with substantially rigid body motion.

In still another aspect, the aerosol generator may further include an end cap that is coupled to the opposite side of the vibratable element. The end cap has a geometry that is similar or identical to the support member. In this way, the aerosol generator is symmetrical about the vibratable element. In some cases, the end cap may have a different shape to facilitate translation of the vibratable member.

In a further aspect, the vibratable element is vibrated at a frequency in the range from about 50 kHz to amount 250 kHz. When vibrated, the vibratable member is displaced a distance in the range from about 0.1 μm to about 10 μm.

### BRIEF DESCRIPTION OF THE DRAWINGS

FIG. 1 is a perspective view of one embodiment of an aerosol generator according to the invention.

FIG. 2 is a cross sectional view of the aerosol generator of FIG. 1 and further illustrating a vibratable member.

FIG. 3 is a cross sectional view of another embodiment of an aerosol generator according to the invention.

FIG. 4 is a schematic diagram of an aerosolization device having the aerosol generator of FIG. 2 according to the invention.

### DESCRIPTION OF THE SPECIFIC EMBODIMENTS

The invention provides aerosol generators and methods for aerosolizing liquids. The aerosol generators are configured such that if operated at the appropriate frequency, such as near or at an appropriate resonant frequency, the aerosol generator accelerates a vibratable member having a plurality of apertures in substantially rigid body motion. In this way, the vibratable member moves back and forth without substantially flexing. In so doing, essentially all of the apertures of the vibratable member eject liquid droplets to increase the efficiency of the aerosol generators and to increase the aerosolization rate. The aerosol generators of the invention may also be operated at other frequencies where some flexing of the vibratable member occurs while still ejecting sufficient liquid droplets.

A variety of aerosol generator configurations may be used to translate the vibratable element back and forth. In one embodiment, the aerosol generator utilizes an annular vibratable element, such as a piezoelectric transducer. The piezoelectric transducer is configured to radially expand and contract when supplied with electric current. Coupled to the piezoelectric transducer is a support member or an end cap that holds the vibratable member. The aerosol generators may include a pair of similar or identical end caps that are attached to opposite sides of the transducer so they are symmetrical about the transducer. This forces the transducer and the overall structure to vibrate in-plane such that the energy is directed into vertically lifting the vibratable member. However, the aerosol generator may function with a single end cap and will vibrate with a different mode. In some cases, one end cap may be somewhat different to accommodate for the addition of the vibratable member on the other end cap, to facilitate the supply of liquids through the end cap, and the like.

A variety of end cap or support member configurations may be utilized. Typically, the end cap has a concave inner surface and a central opening over which the vibratable member is secured. The outer perimeter of the end cap is coupled to the transducer so that as the transducer radially expands and contracts, the vibratable member is raised and lowered.

One partial configuration of the end cap is a cymbal shaped end cap. The cymbal shaped end cap operates in a mode shape such that the radial dilation mode of the piezoelectric ring is mechanically amplified by the mode shape of the cymbal. The radial input from the transducer causes flexure in the curved transition sections of the end caps, thus pushing the center (having the vibratable member) vertically upward as a rigid body. Further, other vibration modes exist that create vibration modes that are similar to those described in U.S. Pat. Nos. 5,164,740; 5,938,117; 5,586,550; 5,758,637 and 6,085,740, incorporated herein by reference. Other end shape configurations are hat shapes and other shapes where the inner region is vertically above the outer region, and a transition region serves as a hinge to move the inner region in an up and down motion.

The cymbal shaped end caps are capable of high force output at a relatively high displacement and frequency to enable high flow rates through the aerosol generator. For example, when operated at frequencies in the range from about 50 kHz to about 250 kHz, displacements in the range from about 0.1 μm to about 10 μm may be achieved.

Another advantage of such end caps is that they may be formed by stamping them from a metal sheet. Examples of metals that may be used include aluminum, stainless steel, brass, polymers, and the like.

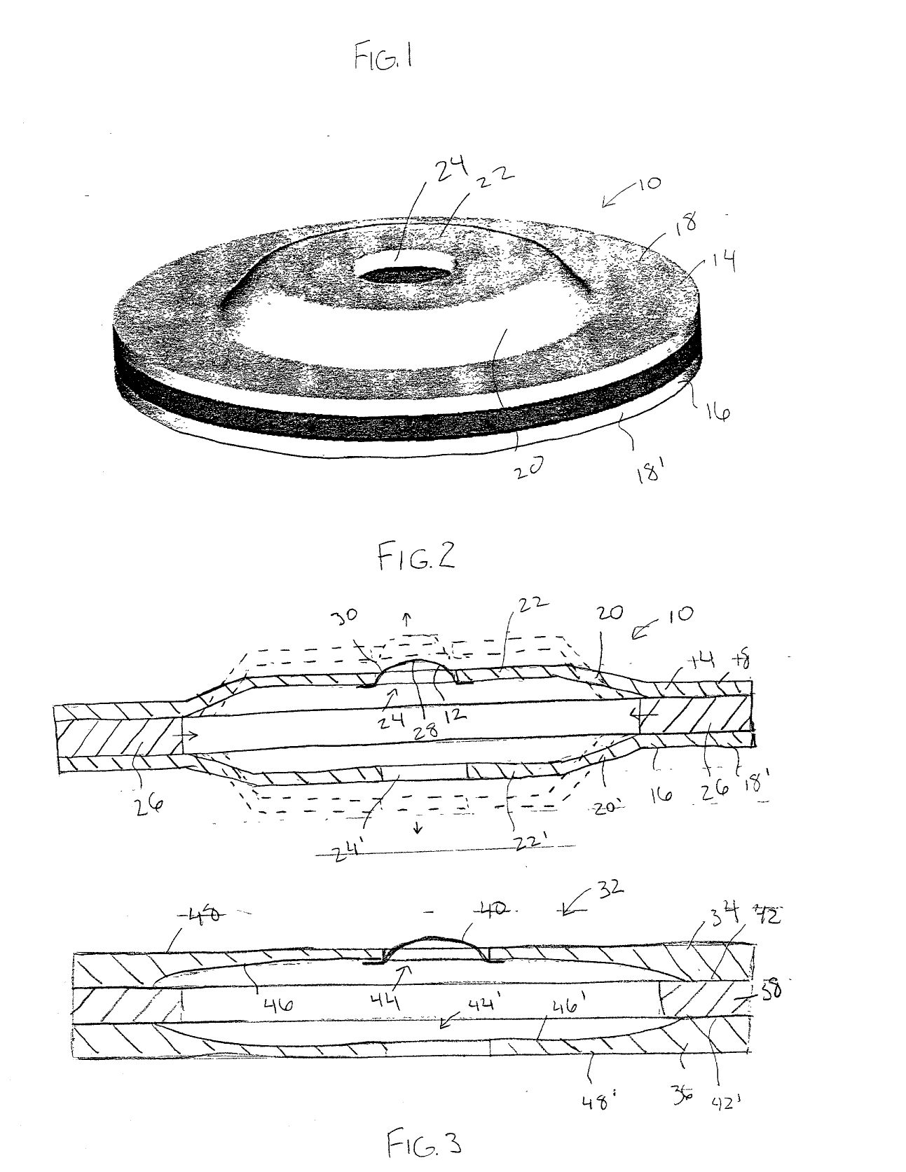

Referring now to FIGS. 1 and 2, one embodiment of an aerosol generator 10 will be described. Aerosol generator 10 utilizes a vibratable member 12 having a plurality of tapered apertures to produce the liquid droplets. For convenience of illustration, vibratable member 12 is omitted from FIG. 1. As shown, vibratable member 12 is dome shaped in geometry and may be similar to those described in U.S. Pat. Nos. 5,586,550; 5,758,637 and 6,085,740, incorporated by reference. However, it will be appreciated that other shapes and configuration may be used, including planar, angled, and the like as well as those described in U.S. Pat. Nos. 5,938,117 and 5,164,740, incorporated herein by reference.

Aerosol generator 10 further includes a pair of cymbal shaped end caps 14 and 16 that have the same shape and size. Optionally, end cap 14 may have a larger opening to hold a liquid reservoir or added features to match the mass of a vibratable member or end cap 14. With aerosol generator 10, end cap 14 serves as a support member to hold vibratable member 12. End cap 14 includes a flat outer region 18, an angled intermediate region 20 and a flat center region 22 that includes a central opening 24. End cap 16 includes similar elements, and for convenience of discussion will be referred to with the same reference numerals, followed by a “′”. Vibratable member 12 is coupled to end cap 14 so as to be held across opening 24.

End caps 14 and 16 are bonded to an annular piezoelectric transducer 26 at outer regions 18 and 18′ as shown in FIG. 2. When electric current is supplied to transducer 26, transducer 26 radially expands and contracts. In so doing, intermediate region 20 serves as a hinge to translate center region 22 up and down. More specifically, when center region 22 lies in an x-y plane, center region 22 moves parallel to a z axis that extends vertically through opening 24. As such, vibratable member 12 is also translated up and down as a generally rigid body, when vibrated at certain modes.

To supply liquid to a rear surface 28 of vibratable member 12, a liquid feed system may be used to introduce liquids through opening 24′. Upon actuation, liquid droplets are ejected from a front surface 30 of vibratable member 12. Although shown with end caps 14 and 16, in some embodiments only end cap 14 may be used, thereby providing greater access to rear surface 28 of vibratable member 12.

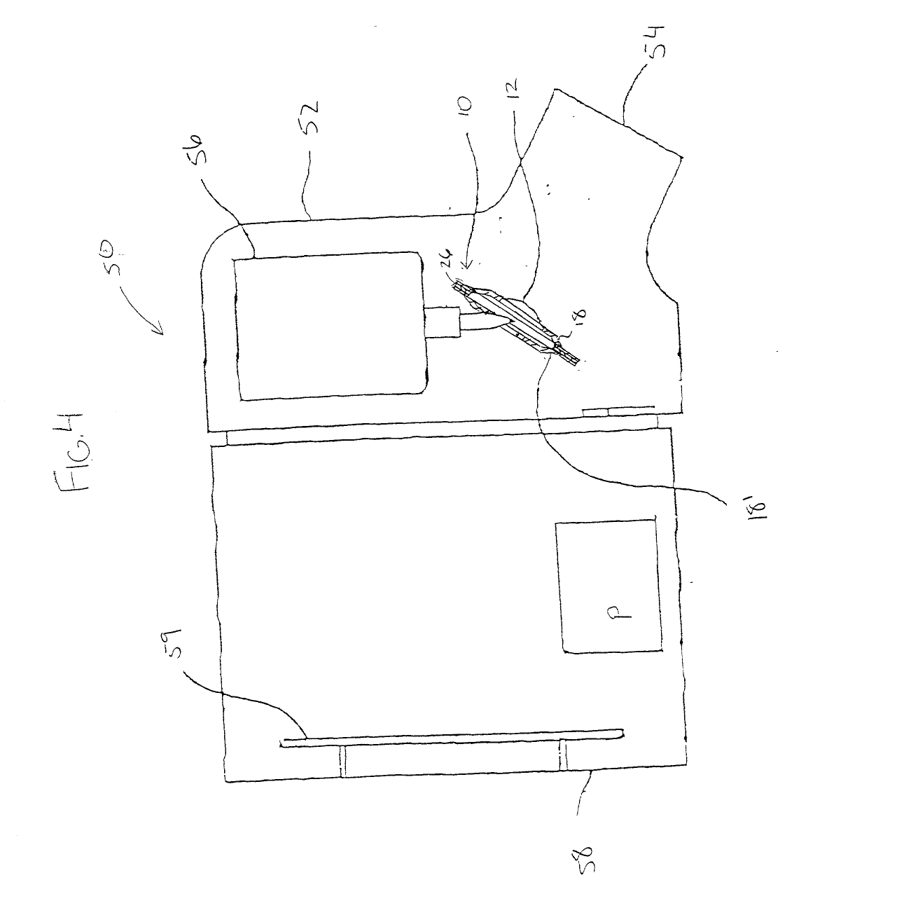

Shown in FIG. 3 is an alternative embodiment of an aerosol generator 32 and comprises a pair of end caps 34 and 36 that are bonded to an annular piezoelectric transducer 38. End cap 34 serves as a support member for a vibratable member 40 having a plurality of apertures. End cap 34 has an outer region 42 that is coupled to transducer 38 and a central opening 44 over which vibratable member 40 is disposed. End cap 34 further includes a concave inner surface 46 and a generally flat top surface 48. End cap 36 includes similar features and are referred to with the same reference numerals, followed by a “′”.

Aerosol generator 32 operates in a manner similar to aerosol generator 10 as previously described. Aerosol generator 32 is illustrated to show that the end caps may have different shapes and different types of concave inner surfaces while still permitting rigid body motion of the vibratable member.

FIG. 4 illustrates one embodiment of an aerosolization device 50 that utilizes aerosol generator 10. However, it will be appreciated that device 50 may utilize any of the aerosol generators described herein. Further, the aerosol generators of the invention are not limited for use with a specific type of aerosolization device, and may be used within a wide variety, including, for example, those described in U.S. Pat. Nos. 5,164,740; 5,938,117; 5,586,550; 5,758,637 and 6,085,740, incorporated herein by reference.

Aerosolization device 50 comprises a housing 52 to hold the various components of aerosolization device 50. Housing 52 further includes a mouthpiece 54 and one or more vents (not shown) to permit air to enter into housing 52 when a user inhales from mouthpiece 54. Disposed within housing 52 is aerosol generator 10 of FIG. 2. However, it will be appreciated that any of the aerosol generators described herein may be placed into housing 52.

Aerosolization device 50 further includes a canister 56 having a supply of liquid that is to be aerosolized by aerosol generator 52. Canister 56 may include a metering valve to place a metered amount of liquid onto aperture plate 12. Although not shown, a button or the like may be employed to dispense the volume of liquid when requested by the user.

Housing 52 includes an electronics region 58 for holding the various electrical components of aerosolization device 50. For example, region 58 may include a printed circuit board 59 which serves as a controller to control operation of the aerosol generator 10. More specifically, circuit board 59 may send (via circuitry not shown) an electrical signal to the piezoelectric element 26 to cause aperture plate 12 to vibrate. A power supply P, such as one or more batteries, is electrically coupled to circuit board 59 to provide aerosolization device 50 with power.

The invention has now been described in detail for purposes of clarity and understanding. However, it will be appreciated that certain changes and modifications may be practiced within the scope of the appended claims.

What is claimed is:

1. An aerosol generator comprising:
a vibratable element having a central opening;
a support member coupled to the vibratable element, the support member having a flat outer region, an angled intermediate region and a flat inner region having a central opening; and
a vibratable member disposed across the central opening of the support member, the vibratable member having a front surface, a rear surface and a plurality of apertures extending therebetween, whereby liquid applied to the rear surface is ejected from the front surface as an atomized spray upon vibration of the vibratable element.

2. An aerosol generator as in claim 1, wherein the vibratable element comprises an annular piezoelectric transducer that is coupled to an outer periphery of the support member so as to be concentric with the vibratable element, and wherein the transducer is configured to radially expand and contract.

3. An aerosol generator as in claim 1, wherein the support member is cymbal shaped in geometry.

4. An aerosol generator as in claim 3, wherein the vibratable element is coupled to the outer region.

5. An aerosol generator as in claim 1, wherein the vibratable element is configured to vibrate at a frequency in the range from about 50 kHz to about 250 kHz, and wherein the support member is configured to move the vibratable member with a displacement in the range from about 0.1 μm to about 10 μm.

6. An aerosol generator as in claim 1, wherein the vibratable element has a first side and a second side, wherein the support member is coupled to the first side, and further comprising an end cap that is coupled to the second side, wherein the end cap has a shape that is similar to that of the support member.

7. An aerosol generator as in claim 1, wherein the vibratable member is dome shaped in geometry and wherein the apertures taper from the rear surface to the front surface.

8. An aerosolization device, comprising:
a housing defining an interior and an exit opening;
an aerosol generator disposed in the interior, the aerosol generator comprising a vibratable element having a central opening; a support member coupled to the vibratable element, the support member having a flat outer region, an angled intermediate region and a flat inner region having a central opening; and a vibratable member disposed across the central opening of the support member, the vibratable member having a front surface, a rear surface and a plurality of apertures extending therebetween; and
circuitry to provide electrical current to the vibratable element to vibrate the vibratable element, whereby liquid applied to the rear surface of the vibratable element is ejected from the front surface as an atomized spray that may exit the housing through the exit opening.

9. A device as in claim 8, further comprising a power supply within the housing to supply power to the circuitry.

10. A device as in claim 8, further comprising a container having a supply of liquid that may be delivered to the rear surface of the vibratable element.

11. A device as in claim 8, wherein the vibratable element comprises an annular piezoelectric transducer that is coupled to an outer periphery of the support member so as to be concentric with the vibratable element, and wherein the transducer is configured to radially expand and contract.

12. A device as in claim 8, wherein the support member is cymbal shaped in geometry.

13. A device as in claim 12, wherein the vibratable element is coupled to the outer region.

14. A device as in claim 8, wherein the vibratable element is configured to vibrate at a frequency in the range from about 50 kHz to about 250 kHz, and wherein the support member is configured to move the vibratable member with a displacement in the range from about 0.1 μm to about 10 μm.

15. A device as in claim 8, wherein the vibratable element has a first side and a second side, wherein the support member is coupled to the first side, and further comprising an end cap that is coupled to the second side, wherein the end cap has a shape that is similar to that of the support member.

16. A device as in claim 8, wherein the vibratable member is dome shaped in geometry and wherein the apertures taper from the rear surface to the front surface.

17. A method for aerosolizing a liquid, the method comprising:
providing an aerosol generator comprising a vibratable element having a central opening; a support member coupled to the vibratable element, the support member having a flat outer region, an angled intermediate region and a flat inner region having a central opening; and a vibratable member disposed across the central opening of the support member, the vibratable member having a front surface, a rear surface and a plurality of apertures extending therebetween;
supplying liquid to the rear surface of the vibratable member; and
vibrating the vibratable element to move the vibratable member and to eject the liquid from the front surface as an atomized spray.

18. A method as in claim 17, further comprising vibrating the vibratable element at a frequency in the range from about 50 kHz to about 250 kHz to cause the vibratable member to move back and forth with a displacement in the range from about 0.1 μm to about 10 μm.

19. A method as in claim 17, wherein the vibratable element comprises an annular piezoelectric transducer that is coupled to an outer periphery of the support member so as to be concentric with the vibratable element, and wherein the transducer is configured to radially expand and contract.

20. A method as in claim 17, wherein the support member is cymbal shaped in geometry.

21. A method as in claim 20, wherein the vibratable element is coupled to the outer region.

22. A method as in claim 17, wherein the vibratable element has a first side and a second side, wherein the support member is coupled to the first side, and further comprising an end cap that is coupled to the second side, wherein the end cap has a shape that is similar to that of the support member.

23. A method as in claim 17, wherein the vibratable member is dome shaped in geometry and wherein the apertures taper from the rear surface to the front surface.

24. An aerosol generator, comprising:
a vibratable element having a central opening;
a support member coupled to the vibratable element, the support member having a flat outer region, an angled intermediate region and a flat inner region having a central opening; and
a vibratable member disposed across the central opening of the support member, the vibratable member having a front surface, a rear surface, and a plurality of apertures extending therebetween;
wherein the support member is configured to move the vibratable member back and forth as a generally rigid body upon vibration of the vibratable element, whereby liquid applied to the rear surface of the vibratable element is ejected from the front surface as an atomized spray.

25. An aerosol generator as in claim 24, wherein the support member defines a generally concave surface and a generally convex surface and wherein the concave surface generally faces the vibratable element.

26. An aerosol generator as in claim 24, wherein the vibratable element comprises an annular piezoelectric transducer that is coupled to an outer periphery of the support member so as to be concentric with the vibratable element, and wherein the transducer is configured to radially expand and contract.

27. An aerosol generator as in claim 25, wherein the support member is cymbal shaped in geometry.

28. An aerosol generator as in claim 27, wherein the vibratable element is coupled to the outer region.

29. An aerosol generator as in claim 24, wherein the vibratable element is configured to vibrate at a frequency in the range from about 50 kHz to about 250 kHz, and wherein the support member is configured to move the vibratable member with a displacement in the range from about 0.1 μm to about 10 μm.

30. An aerosol generator as in claim 25, wherein the vibratable element has a first side and a second side, wherein the support member is coupled to the first side, and further comprising an end cap that is coupled to the second side, wherein the end cap has a shape that is similar to that of the support member.

31. An aerosol generator as in claim 24, wherein the vibratable member is dome shaped in geometry and wherein the apertures taper from the rear surface to the front surface.

32. A method for aerosolizing a liquid, the method comprising:
providing an aerosol generator comprising a support member coupled to the vibratable element, the support member having a central opening; and
a vibratable member disposed across the central opening of the support member, the vibratable member having a front surface, a rear surface, and a plurality of apertures extending therebetween;
supplying liquid to the rear surface of the vibratable member; and
vibrating the vibratable element to move the vibratable member back and forth in a substantially rigid body motion and thereby eject the liquid from the front surface as an atomized spray.

33. A method as in claim 32, further comprising vibrating the vibratable element at a frequency in the range from about 50 kHz to about 250 kHz to cause the vibratable member to move back and forth with a displacement in the range from about 0.1 μm to about 10 μm.

34. A method as in claim 32, wherein the support member has a generally concave surface and a generally convex surface and wherein the concave surface generally faces the vibratable element.

35. A method as in claim 32, wherein the vibratable element comprises an annular piezoelectric transducer that is coupled to an outer periphery of the support member so as to be concentric with the vibratable element, and wherein the transducer is configured to radially expand and contract.

36. A method as in claim 35, wherein the support member is cymbal shaped in geometry.

37. A method as in claim 34, wherein the vibratable element has a first side and a second side, wherein the support member is coupled to the first side, and further comprising an end cap that is coupled to the second side, wherein the end cap has a shape that is similar to that of the support member.
